(19) United States
(12) Patent Application Publication
(10) Pub. No.: US 2012/0115802 A1
(43) Pub. Date: May 10, 2012

(54) Method for using ulipristal acetate with cytochrome isozyme modulators

(75) Inventor: Erin Gainer, Paris (FR)

(73) Assignee: LABORATOIRE HRA PHARMA, Paris (FR)

(21) Appl. No.: 13/104,759

(22) Filed: May 10, 2011

Related U.S. Application Data
(60) Provisional application No. 61/333,564, filed on May 11, 2010.

(51) Int. Cl. A61K 31/57; A61P 35/00; A61P 15/00; A61P 5/00; A61P 15/18; A61K 31/7048

(52) U.S. Cl. 514/29; 514/178; 514/171

(57) ABSTRACT

The invention relates to a method of using ulipristal acetate or a metabolite thereof for providing contraception or for treating a patient's condition, comprising providing a patient with ulipristal acetate or a metabolite thereof, and informing the patient or a medical care worker that ulipristal acetate or a metabolite thereof affects activity of a cytochrome p450 isozyme, and that administration of ulipristal acetate or a metabolite thereof with a substance that affects activity of a cytochrome p450 isozyme can affect plasma concentration, safety, efficacy or any combination thereof of ulipristal acetate or a metabolite thereof, the substance, or both.

### CROSS REFERENCE TO RELATED APPLICATION

This application claims priority under 35 USC §119(e) to U.S. provisional patent application Ser. No. 61/333,564 filed May 11, 2010.

The present invention relates to improved methods of using ulipristal acetate.

### BACKGROUND TO THE INVENTION

Ulipristal acetate, formerly known as CDB-2914, and methods for its preparation, are described e.g., in U.S. Pat. Nos. 4,954,490; 5,073,548, and 5,929,262, as well as in international patent applications WO2004/065405 and WO2004/078709. Ulipristal acetate possesses antiprogestational and antiglucocorticoidal activity, and has been proposed for contraception, in particular for emergency contraception, and for the therapy of various hormonal diseases. Properties of this compound are further described in Blithe et al, Steroids. 2003 68(10-13):1013-7.

In a double-blind non-inferiority trial, ulipristal acetate was shown to be as efficacious as levonorgestrel for preventing pregnancy when used within 72 hours of unprotected intercourse (Creinin et al, Obstetrics & Gynecology, 2006, Vol. 108; No. 5: 1089-97). Ulipristal acetate has been approved in Europe, under trademark EllaOne®, for use as an emergency contraceptive.

Studies directed to possible interactions of ulipristal acetate with other active agents have been limited. There have been no detailed studies of the specific enzymes involved in metabolism of or of the inhibitory or inducing effects of ulipristal acetate on any Phase I or Phase II metabolic enzymes.

Several major enzymes and pathways are involved in drug metabolism. Pathways of drug biotransformation are usually divided into two major groups of reactions: Phase I and Phase II metabolism.

Some typical examples of Phase I metabolism include oxidation, hydrolysis and reduction. Examples of Phase I enzymes involved in oxidation reactions are the cytochrome p450 monooxygenase system, the flavin-containing monooxygenase system, alcohol dehydrogenase and aldehyde dehydrogenase, monoamine oxidase, and peroxidases for co-oxidation. Examples of Phase I enzymes involved in reduction are NADPH-cytochrome p450 reductase and reduced (ferrous) cytochrome p450. Examples of Phase I hydrolysis enzymes are epoxide hydrolase, esterases and amidases.

Phase II metabolism involves conjugation reactions. Typical conjugation reactions are glucuronidation, sulfation, amino acid conjugation, acetylation, methylation, and mercapturic acid conjugation. Examples of Phase II metabolic enzymes are glutathione S-transferases (GSTs), mercapturic acid biosynthetic enzymes (transpeptidases, peptidases, and N-acetylases), UDP-glucoron(os)yltransferases, N-acetyltransferases, amino acid N-acyl transferases, and sulfotransferases.

One of the most important groups of Phase I enzymes are the cytochrome p450 monooxygenase system enzymes. The cytochrome p450 enzymes are a highly diverse superfamily of enzymes. NADPH is required as a coenzyme and oxygen is used as a substrate. Each enzyme is termed an isoform or isozyme since each derives from a different gene.

By understanding the unique functions and characteristics of Phase I and Phase II metabolic enzymes, physicians may better anticipate and manage active agent interactions and may predict or explain an individual's response to a particular therapeutic regimen.

There accordingly remains a need in the art for improved methods for the administration and use of ulipristal acetate, in particular methods that take into account the effects of ulipristal acetate on activity of Phase I and Phase II metabolic enzymes, including the cytochrome P450 isozymes.

### SUMMARY OF THE INVENTION

The inventors have found out that ulipristal acetate or a metabolite thereof is metabolized by cytochrome p450 (CYP) isozymes in liver microsomes in vitro. As a result, co-administration with CYP inhibitors increases exposure to ulipristal acetate or a metabolite thereof. Conversely, co-administration with CYP inducers could reduce plasma concentration of ulipristal acetate or a metabolite thereof and could result in a decrease in effectiveness.

On this basis, the invention provides a method of using ulipristal acetate or a metabolite thereof for providing contraception, or for treating a patient's condition, comprising providing a patient with ulipristal acetate or a metabolite thereof, and informing the patient or a medical care worker that ulipristal acetate or a metabolite thereof affects activity of a cytochrome p450 isozyme, and that administration of ulipristal acetate or a metabolite thereof with a substance that affects activity of a cytochrome p450 isozyme can affect plasma concentration, safety, effectiveness or any combination thereof of ulipristal acetate or a metabolite thereof, the substance, or both.

Preferably it is provided a method of using ulipristal acetate or a metabolite thereof to provide contraception or to treat a patient's condition, comprising: providing a patient with ulipristal acetate or a metabolite thereof; and informing the patient or a medical care worker that a cytochrome p450 isozyme metabolizing ulipristal acetate or a metabolite thereof is CYP3A4 and that administration of ulipristal acetate or a metabolite thereof and a substance that is a substrate, inhibitor, or inducer of CYP34A can affect plasma concentration, safety, effectiveness or any combination thereof of ulipristal acetate or a metabolite thereof, the substance, or both.

In a particular embodiment, the invention provides a method for providing contraception, preferably post coital contraception, in a female subject, comprising providing the subject with an effective amount of ulipristal acetate or a metabolite thereof, in combination with an inhibitor of CYP3A4 enzyme.

Another subject of the invention is a kit comprising i) a dosage form comprising ulipristal acetate or a metabolite thereof and ii) a dosage form comprising an inhibitor of CYP3A4 enzyme.

Still another subject of the invention is a pharmaceutical composition comprising ulipristal acetate or a metabolite thereof and an inhibitor of CYP3A4 enzyme, in association with a pharmaceutically acceptable carrier.

A subject of the invention is a kit comprising i) a dosage form comprising ulipristal acetate or a metabolite thereof, and (ii) printed matter stating that administration of ulipristal acetate or a metabolite thereof with a substance that affects activity of a cytochrome p450 isozyme can affect plasma concentration, safety, effectiveness or any combination thereof of ulipristal acetate or a metabolite thereof, the substance, or both

A particular subject of the invention is a kit comprising i) a dosage form comprising ulipristal acetate or a metabolite thereof, and (ii) printed matter stating that administration of an inducer of CYP3A4 enzyme may decrease plasma concentration of ulipristal acetate or a metabolite thereof, and/or that administration of an inducer of CYP3A4 enzyme may decrease effectiveness of ulipristal acetate or a metabolite thereof.

It is further described a method of using ulipristal acetate, comprising, administering ulipristal acetate to a patient,

determining if the patient is taking an inhibitor or inducer of a Cytochrome enzyme such as CYP3A4,
determining side effects, e.g. by measuring the plasma concentration, the efficiency profile, or the safety profile of ulipristal acetate, and
adjusting dosing of ulipristal acetate or of inhibitor or inducer of the Cytochrome enzyme if at least one toxic side effect is detected.

### DETAILED DESCRIPTION OF THE INVENTION

### Ulipristal Acetate

Ulipristal acetate, formerly known as CDB-2914, designates within the context of this application 17α-acetoxy-11β-[4-N,N-dimethylamino-phenyl)-19-norpregna-4,9-diene-3,20-dione, represented by formula I:

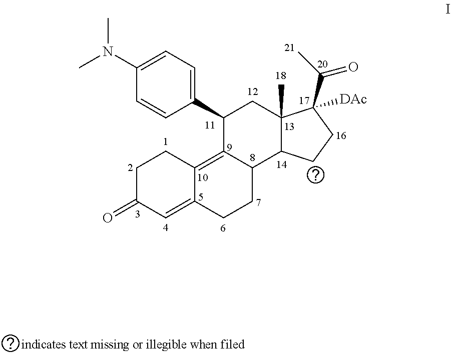

Metabolites of CDB-2914, include those described in Attardi et al, 2004, al, Journal of Steroid Biochemistry & Molecular Biology, 2004, 88: 277-288,

e.g. monodemethylated CDB-2914 (CDB-3877); didemethylated CDB-2914 (CDB-3963); 17alpha-hydroxy CDB-2914 (CDB-3236); aromatic A-ring derivative of CDB-2914 (CDB-4183).

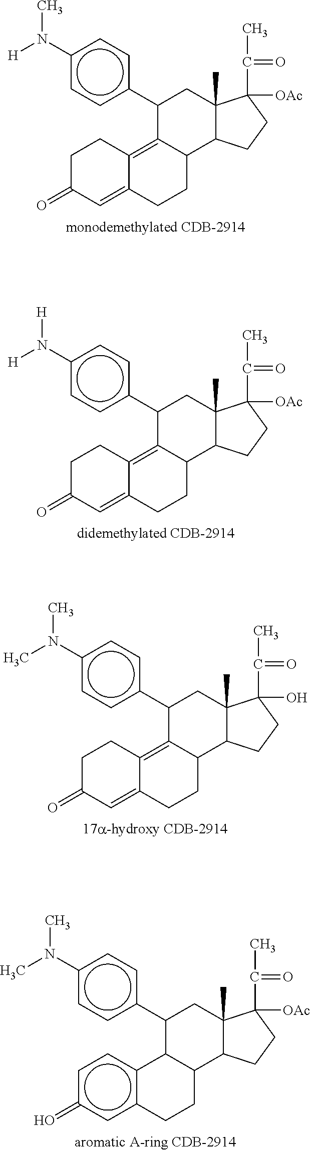

The main metabolite is monodemethylated CDB-2914 (CDB-3877A), that is 17α-acetoxy-11β-[4-N-methylamino-phenyl)-19-norpregna-4,9-diene-3,20-dione.

### Indications:

Ulipristal acetate is useful in a number of therapeutic indications, including contraception, including emergency contraception.

Other indications include, but are not limited to, treatment of conditions such as endometriosis, dysmenorrhea, uterine leiomyoma (leiomyomata), uterine fibroid, excessive uterine bleeding (menorrhagia), either idiopathic or resulting from spontaneous or iatrogenic coagulation disorders, meningioma, hormonal diseases, such as hormone-responsive cancers, endocrine hormone-dependent tumors, breast cancer and inhibition of uterine endometrial proliferation.

In a particular embodiment, the subject, who may be also designated by the term “patient”, may be any woman in need of contraception, preferably of a post-coital contraception, preferably an emergency contraception.

Any woman of reproductive age may need post-coital or emergency contraception at some point to avoid an unintended pregnancy. It is meant to be used in situations of unprotected intercourse, such as:

when no contraceptive has been used;
when there is a contraceptive failure or incorrect use, including:
condom breakage, slippage, or incorrect use;
non-compliance with dosage regimen for combined oral contraceptive pills;
non-compliance with dosage regimen for progestogen-only pill (minipill);
more than two weeks late for a progestogen-only contraceptive injection (depot-medroxyprogesterone acetate or norethisterone enanthate);
more than seven days late for a combined estrogen-plus-progestogen monthly injection;
dislodgment, delay in placing, or early removal of a contraceptive hormonal skin patch or ring;
dislodgment, breakage, tearing, or early removal of a diaphragm or cervical cap;
failed coitus interruptus (e.g., ejaculation in vagina or on external genitalia);
failure of a spermicide tablet or film to melt before intercourse;
miscalculation of the periodic abstinence method or failure to abstain on fertile day of cycle;
IUD expulsion; or in cases of sexual assault when the woman was not protected by an effective contraceptive method.

Preferably post coital contraception is provided within 120 hours, preferably within 72 hours, after unprotected intercourse. For instance, post coital contraception may be provided more than about 2, 3, 4 and up to 5 or even 6 days after unprotected intercourse. Preferably, post coital contraception is provided within about 75, 80, 90, or 96 hours after unprotected intercourse. Post coital contraception may be provided up to 120 hours, preferably about 100, 110, 120 hours after unprotected intercourse.

In the present invention post coital contraception most preferably is an emergency contraception.

### Routes of Administration:

Ulipristal acetate or a metabolite thereof may be administered by any convenient route, including oral, buccal, parenteral, transdermal, vaginal, uterine, rectal, etc.

For a brief review of present methods for drug delivery, see, Langer, Science 249:1527-1533 (1990), which is incorporated herein by reference. Methods for preparing administrable compounds are known or are apparent to those skilled in the art and are described in more detail in, for example, Remington's Pharmaceutical Science, 17th ed., Mack Publishing Company, Easton, Pa. (1985), which is incorporated herein by reference, and which is hereinafter referred to as “Remington.”

For solid compositions, conventional nontoxic solid carriers may be used which include, for example, pharmaceutical grades of mannitol, lactose, starch, magnesium stearate, sodium saccharine, talcum, cellulose, glucose, sucrose, magnesium, carbonate, and the like. For oral administration, a pharmaceutically acceptable nontoxic composition is formed by incorporating any of the normally employed excipients, such as those carriers previously listed.

Oral solid dosage forms preferentially are compressed tablets or capsules. Compressed tablets may contain any of the excipients described above which are diluents to increase the bulk of the ulipristal so that production of a compressed tablet of practical size is possible. Binders, which are agents which impart cohesive qualities to powdered materials are also necessary. Starch, gelatin, sugars such as lactose or dextrose, and natural and synthetic gums are used. Disintegrants are necessary in the tablets to facilitate break-up of the tablet. Disintegrants include starches, clays, celluloses, algins, gums and crosslinked polymers. Lastly small amounts of materials known as lubricants and glidants are included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. Procedures for the production and manufacture of compressed tablets are well known by those skilled in the art (See Remington).

Capsules are solid dosage forms using preferentially either a hard or soft gelatin shell as a container for the mixture of ulipristal or a metabolite thereof and inert ingredients. Procedures for production and manufacture of hard gelatin and soft elastic capsules are well known in the art (See Remington).

Buccal forms or devices are also useful, such as those described in U.S. patent application 20050208129, herein incorporated by reference. U.S. patent application 20050208129 describes a prolonged release bioadhesive mucosal therapeutic system containing at least one active principle, with an active principle dissolution test of more than 70% over 8 hours and to a method for its preparation. Said bioadhesive therapeutic system comprises quantities of natural proteins representing at least 50% by weight of active principle and at least 20% by weight of said tablet, between 10% and 20% of a hydrophilic polymer, and compression excipients, and comprising between 4% and 10% of an alkali metal alkylsulphate to reinforce the local availability of active principle and between 0.1% and 1% of a monohydrate sugar.

For parenteral administration, fluid unit dosage forms are prepared utilizing the compounds and a sterile vehicle, water being preferred. Ulipristal acetate or a metabolite thereof, depending on the vehicle and concentration used, can be either suspended or dissolved in the vehicle. In preparing solutions the compound can be dissolved in water for injection and filtered sterilized before filling into a suitable vial or ampoule and sealing. Advantageously, adjuvants such as a local anesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection is supplied to reconstitute the liquid prior to use. Parenteral suspensions can be prepared in substantially the same manner except that the compounds are suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of ulipristal acetate or a metabolite thereof.

Additionally, a suppository can be employed to deliver ulipristal acetate or a metabolite thereof. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate. These suppositories can weigh from about 1 to 2.5 g.

Transdermal delivery systems comprising a penetration enhancer and an occlusive backing are of use to deliver ulipristal acetate or a metabolite thereof. Examples of penetration enhancers include dimethyl sulfoxide, dimethyl acetamide and dimethylformamide.

Systems comprising polymeric devices which slowly release or slowly erode and release within the body to provide continuous supplies of ulipristal acetate or a metabolite thereof are also of use. Suitable delivery systems include subcutaneous devices or implants such as those routinely used to deliver norgestrienone or progestin R2323 and other medicaments.

Ulipristal acetate or a metabolite thereof is preferably in form of an oral dosage, such as a tablet or a capsule, preferably a tablet.

In a preferred embodiment, it is provided as pharmaceutical tablet for oral administration, comprising ulipristal acetate or a metabolite thereof in an amount of 3 to 18 wt %, together with the following excipients: a diluent in an amount of 60 to 95 wt %, a binding agent in an amount of 1 to 10 wt %, croscarmellose sodium in an amount of 1 to 10 wt %, and magnesium stearate in an amount of 0 to 5 wt %.

According to preferred embodiments, the composition, preferably in form of a tablet, comprises 10% wt ulipristal acetate or a metabolite thereof and is designed to contain from about 5 to about 50 mg ulipristal acetate or a metabolite thereof, preferably about 10, 20, or 30 mg.

The diluent may be selected from any pharmaceutically acceptable agent or combination of agents that increases the bulk quantity of ulipristal acetate or a metabolite thereof so that production of a compressed tablet of practical size is possible. In a preferred embodiment, the diluent is selected from the group consisting of a monosaccharide, a disaccharide, a derivative polyol of a monosaccharide and hydrates thereof. The term ‘derivative polyol of a monosaccharide’ stands for a sugar alcohol such as mannitol, xylitol or sorbitol. Preferably the diluent is selected from the group consisting of lactose monohydrate and mannitol. In a most preferred embodiment, the diluent is lactose monohydrate is an amount of 65 to 92 wt %, more preferably 70-85 wt %.

The binding agent, or binder, may be selected from any pharmaceutically acceptable agent (or combination of agents) which imparts cohesive qualities to powdered materials. The binding agent may be selected from starch, gelatin, sugars such as cellulose derivatives, and natural and synthetic gums may be used. Advantageously, the binding agent of the tablet is selected from the group consisting of polymers. The binding agent may be a natural polymer material such as polysaccharide, or a synthetic polymer such as a plastic polymer. Preferably, the binding agent is hydroxypropyl methyl cellulose and/or povidone. In a preferred embodiment, the binding agent is or comprises povidone, preferably 1.5% to 8.5 wt % of povidone, even more preferably between 3-7 wt %, most preferably about 5 wt % povidone.

The tablets preferably comprise croscarmellose sodium. Croscarmellose sodium is a disintegrant, e.g., facilitates break-up of the tablet. Croscarmellose sodium may be used alone or in combination with other disintegrants, preferably alone. It is preferably present in an amount of 1 to 10 wt/%, preferably 1.5 to 8.5 wt %, and more preferably 4.5 to 5.5 wt %, or even more preferably about 5 wt %.

In preferred embodiments, the tablets of the present invention contain magnesium stearate. While magnesium stearate may be used in combination with other lubricants, it is preferably used alone, in an amount comprised between 0.5 and 5 wt %.

Preferably, the tablet according to the present invention comprises lactose monohydrate as a diluent and povidone as a binding agent.

In a more specific embodiment, the tablet comprises: ulipristal acetate or a metabolite thereof 5 to 15 wt %, lactose monohydrate 71 to 87 wt %, povidone 4.5 to 5.5 wt %, croscarmellose sodium 4.5 to 5.5 wt % and magnesium stearate 1 to 4 wt %, where the total percentage adds up to 100.

In an even more specific embodiment, the tablet comprises: ulipristal acetate or a metabolite thereof 10%, lactose monohydrate 79 wt %, povidone 5 wt %, croscarmellose sodium 5 wt % and magnesium stearate 1 wt %.

Tablets may be prepared according to techniques known per se in the art. Suitable methods include direct compression (“dry blending”), dry granulation followed by compression, and wet granulation followed by drying and compression. Several methods include the use of compacting roller technology such as a chilsonator or drop roller, or molding, casting, or extrusion technologies. The tablet can be a coated tablet or an uncoated tablet.

In the preparation of the tablets, commercial mixtures comprising diluents and binding agents may be used, such as Avicel® (microcristalline cellulose), Starlac® (lactose monohydrate 85% with maize starch 15%) or, Ludipress® (lactose monohydrate 93% with Povidone 7%).

In a particular embodiment, a 30 mg ulipristal acetate tablet may be manufactured as follows. Lactose monohydrate 79 wt %, ulipristal acetate 10 wt % and povidone 5 wt % are mixed and purified water is added. This granulation step is followed by a drying step in an oven at 40° C. Croscarmellose sodium 5 wt % and magnesium stearate 1 wt % are added for the lubrication step. The obtained formulation is compressed to get the tablet, which shows the following formulation (Table 1).

TABLE 1
30 mg ulipristate acetate tablet:

| | Quantity for one | Quantity for one |
|---|---|---|
| Ingredients | tablet (mg) | tablet (wt %) |
| Ulipristal acetate | 30.00 | 10 |
| Lactose Monohydrate | 237.00 | 79 |
| Povidone | 15.00 | 5 |
| Croscarmellose sodium | 15.00 | 5 |
| Magnesium stearate | 3.00 | 1 |
| Total | 300.00 | 100 |

Further ulipristal acetate tablets are provided hereafter.

TABLE 2
Other ulipristal acetate tablet formulations:

| | 10 mg tablet | 30 mg tablet |
|---|---|---|
| | Quantity for one | Quantity for one |
| Ingredients | tablet in mg (wt %) | tablet in mg (wt %) |
| Ulipristal acetate | 10.00 (10) | 30.00 (10) |
| Lactose Monohydrate | 79.00 (79) | 246.00 (82) |
| Povidone | 5.00 (5) | 9.00 (3) |
| Croscarmellose sodium | 5.00 (5) | 12.00 (4) |
| Magnesium stearate | 1.00 (1) | 3.00 (1) |
| Total | 100.00 (100) | 300.00 (100) |

Substances that Affect Cytochrome P 450 Activity:

Cytochrome P-450 is a superfamily of enzymes that metabolize a large number of drugs, xenobiotics and endogenous substances in vitro and in vivo. Enzymes of the cytochrome P450 superfamily catalyze the oxidative metabolism of a variety of substrates, including natural compounds such as steroids, fatty acids, prostaglandins, leukotrienes, and vitamins, as well as drugs, carcinogens, mutagens, and xenobiotics.

Changes in active agent metabolism due to competition for the same cytochrome p450 isoform can change the clinical effectiveness or safety of an active agent by altering the plasma concentration of the active agent or its metabolite(s). Similarly, inhibition or induction of the cytochrome p450 isoform that metabolizes a particular active agent can change the clinical effectiveness or safety of that active agent. Therefore, for any cytochrome p450 for which ulipristal acetate acts as a substrate, inhibitor, or inducer, the administration of ulipristal acetate or a metabolite thereof with a substance that is a substrate, inhibitor, or inducer of that cytochrome p450 can affect the metabolism of the ulipristal acetate or a metabolite thereof or the substance.

In one embodiment, the method comprises informing a user that ulipristal acetate or a metabolite thereof inhibits activity of a cytochrome p450 isozyme. The cytochrome p450 isozyme may be any cytochrome p450 isozyme. For example the cytochrome p450 isozyme may be CYP2C9, CYP2D6, or CYP3A4. In preferred embodiments the cytochrome p450 isozyme is a human enzyme. In some embodiments, the method further comprises providing the user with ulipristal acetate or a metabolite thereof.

Informing the user that ulipristal acetate or a metabolite thereof affects the activity of a cytochrome p450 isozyme includes providing a user with information about any effect of ulipristal acetate or a metabolite thereof on the activity of any cytochrome p450 isozyme. Informing the user that ulipristal acetate or a metabolite thereof affects the activity of a cytochrome p450 isozyme includes informing a user of any of the following: that ulipristal acetate is metabolized by a cytochrome p450 isozyme; that a cytochrome p450 isozyme metabolizing ulipristal acetate is CYP3A4; that ulipristal acetate is a inhibitor of CYP3A4; that ulipristal acetate is a substrate of CYP3A4; that there is a potential active agent interaction between ulipristal acetate and an active agent that is a substrate, inhibitor, or inducer of CYP2C9, CYP2D6, or CYP3A4; that ulipristal acetate is a weak inhibitor of a cytochrome p450 isozyme; that caution is recommended when ulipristal acetate or a metabolite thereof and a substrate of CYP2C9, CYP2D6, or CYP3A4 are administered to a patient known to have a poor metabolizer phenotype for or that has reduced activity of CYP2C9, CYP2D6, or CYP3A4; that caution is recommended when administering ulipristal acetate or a metabolite thereof with the substance when the substance is an active agent having a narrow therapeutic index; that the allelic variants of CYP2C9, CYP2D6, or CYP3A4 present in the patient can further affect the potential active agent interaction between ulipristal acetate or a metabolite thereof and an active agent; that there is a potential active agent interaction of ulipristal acetate or a metabolite thereof with an active agent that is a substrate of the cytochrome p450 isozyme; that there is a potential active agent interaction of ulipristal acetate or a metabolite thereof with warfarin; that ulipristal acetate or a metabolite thereof affects the activity of CYP2C9, CYP2D6, or CYP3A4; that there is a potential active agent interaction of ulipristal acetate or a metabolite thereof with a substance that is a substrate of CYP2C9, CYP2D6, or

CYP3A4; that ulipristal acetate or a metabolite thereof is an inhibitor of CYP2C9, CYP2D6, or CYP3A4.

The method can further comprise informing the user that administration of ulipristal acetate or a metabolite thereof with a substance can affect the plasma concentration, bioavailability, safety, efficacy, or a combination comprising at least one of the foregoing of ulipristal acetate or a metabolite thereof or the substance. In some embodiments, the method further comprises providing the user with the substance.

The effect of coadministration of ulipristal acetate or a metabolite thereof and the substance can be determined by comparison of the plasma concentration, bioavailability, safety, efficacy, or a combination comprising at least one of the foregoing of the substance with and without coadministration of ulipristal acetate or a metabolite thereof or by comparison of the plasma concentration, bioavailability, safety, efficacy, or a combination comprising at least one of the foregoing of ulipristal acetate or a metabolite thereof with and without coadministration of the substance.

Examples of substances that are substrates of CYP2C9 include diclofenac, ibuprofen, meloxicam, S-naproxen, piroxicam, suprofen, tolbutamide, glipizide, losartan, irbesartan, glyburide (glibenclamide), glipizide, glimepiride, amitriptyline, celecoxib, fluoxetine, fluvastatin, nateglinide, phenyloin, rosiglitazone, tamoxifen, torsemide, and S-warfarin.

Examples of substances that are substrates of CYP2D6 include carvedilol, S-metoprolol, propafenone, timolol; amitriptyline, clomipramine, desipramine, imipramine, paroxetine; haloperidol, perphenazine, risperidone, thioridazine; alprenolol, amphetamine, aripiprazole, atomoxetine, bufuralol, chlorpheniramine, chlorpromazine, codeine, debrisoquine, dexfenfluramine, dextromethorphan, duloxetine, encamide, flecamide, fluoxetine, fluvoxamine, lidocaine, metoclopramide, methoxyamphetamine, mexiletine, minaprine, nebivolol, nortriptyline, ondansetron, perhexyline, phenacetin, phenformin, propranolol, sparteine, tamoxifen, tramadol, and venlafaxine.

Examples of substrates of CYP3A4 include clarithromycin, erythromycin, telithromycin: quinidine; alprazolam, diazepam, midazolam, triazolam; cyclosporine, tacrolimus (FK506); indinavir, nelfinavir, ritonavir, saquinavir; cisapride; astemizole, chlorpheniramine, terfenadine; amlodipine, diltiazem, felodipine, lercanidipine, nifedipine, nisoldipine, nitrendipine, verapamil; atorvastatin, cerivastatin, lovastatin, simvastatin; estradiol, hydrocortisone, progesterone, testosterone; alfentanyl, aripiprazole, buspirone, cafergot, caffeine, cilostazol, cocaine, codeine, dapsone, dextromethorphan, docetaxel, domperidone, eplerenone, fentanyl, finasteride, gleevec, haloperidol, irinotecan, Levo-Alpha Acetyl Methadol (LAAM), lidocaine, methadone, nateglinide, odanestron, pimozide, propranolol, quinine, salmeterol, sildenafil, sirolimus, tamoxifen, taxol, terfenadine, trazodone, vincristine, zaleplon, and zolpidem.

In a preferred embodiment, the substrate may be an inhibitor of CYP3A4 enzyme such ketoconazole, itraconazole, ritonavir, telitromycin, clarithromycin or nefazodone.

CYP3A4 inducers include carbamazepine, phenobarbital, phenyloin, dexamethasone and other glucocorticoids; barbiturates, various steroids, antibiotics such as rifampin, rifabutin, erythromycin; phenylbutazone, sulfadimidine, sulfinpyrazone, troleandomycin substrates include: benzodiazepines such as alprazolam, diazepam, midazolam, and triazolam; immune modulators such as cyclosporine; antihistamines such as astemizole and chlorpheniramine; HMG CoA Reductase inhibitors such as atorvastatin, cerivastatin, lovastatin, and simvastatin; channel blockers such as diltiazem, felodipine, nifedipine, nisoldipine, nitrendipine, and verapamil; antibiotics such as clarithromycin, erythromycin, and rapamycin; various steroids including cortisol, testosterone, progesterone, estradiol, ethinylestradiol, hydrocortisone, prednisone, and prednisolone; acetominophen, aldrin, alfentanil, amiodarone, astemizole, benzphetamine, budesonide, carbemazepine, cyclophosphamide, ifosphamide, dapsone, digitoxin, quinidine (anti-arrhythmic), etoposide, flutamide, imipramine, lansoprazole, lidocaine, losartan, omeprazole, retinoic acid, FK506 (tacrolimus), tamoxifen, taxol, teniposide, terfenadine, buspirone, haloperidol (antipsychotic), methadone, sildenafil, trazodone, theophylline, toremifene, troleandomycin, warfarin, zatosetron, zonisamide. Particularly potent CYP3A4 inducers include rifampicin, phenyloin, Phenobarbital, carbamazepine, Hypericum perforatrum (St John's wort).

### Combinations:

According to the invention, ulipristal acetate or a metabolite thereof may be combined with an inhibitor of a cytochrome p450 isozyme. The cytochrome p450 isozyme may be any cytochrome p450 isozyme. For example the cytochrome p450 isozyme may be CYP2C9, CYP2D6, or CYP3A4, especially CYP3A4. Examples of such inhibitors are described above.

Ulipristal acetate or a metabolite thereof and the inhibitor may be administered to a patient simultaneously, or subsequently. They may be combined within the same pharmaceutical composition, or they may be presented in different compositions, and administered by the same or by a different route of administration.

In a particular embodiment, it is provided a kit comprising (i) a dosage form comprising ulipristal acetate or a metabolite thereof, and (ii) a dosage form comprising an inhibitor of inhibitor of a cytochrome p450 isozyme, such as an inhibitor of CYP3A4.

Such combinations are useful in any therapeutic indication as described above, including contraception, including emergency contraception.

Other indications include, but are not limited to, treatment of conditions such as endometriosis, dysmenorrhea, uterine leiomyoma (leiomyomata), uterine fibroid, excessive uterine bleeding (menorrhagia), either idiopathic or resulting from spontaneous or iatrogenic coagulation disorders, meningioma, hormonal diseases, such as hormone-responsive cancers, endocrine hormone-dependent tumors, breast cancer and inhibition of uterine endometrial proliferation.

The invention provides a method for providing contraception, or treating a patient's condition such as any condition recited above, which method comprises administering the patient with an effective amount of ulipristal acetate or a metabolite thereof, in combination with an inhibitor of a cytochrome p450 isozyme, such as an inhibitor of CYP3A4.

In a preferred embodiment, it is provided a method for providing post-coital contraception, which method comprises administering the patient with an effective amount of ulipristal acetate or a metabolite thereof, in combination with an inhibitor of CYP3A4 enzyme.

The inhibitor inhibits the removal of ulipristal acetate or a metabolite thereof by the cytochrome isozyme, thus allowing treatment of the condition or achieving contraception using lower amounts of ulipristal acetate or a metabolite thereof than the treatment using ulipristal acetate or a metabolite thereof in the absence of such inhibitor. For instance, one may use a dosage of less than 30 mg ulipristal acetate or a metabolite thereof, preferably 20 mg, preferably 10 mg, or 5 mg, once or twice per day.

In one embodiment, the cytochrome p450 isozyme inhibitor is administered before the ulipristal acetate or a metabolite thereof administration, for example, starting at least one day prior to the administration of ulipristal acetate or a metabolite thereof. In one embodiment, the inhibitor is administered daily starting at least one day prior to the ulipristal acetate or a metabolite thereof administration, and is continued with daily administration during the administration of the ulipristal acetate or a metabolite thereof.

The administration of the inhibitor can be continued for one or more days after completion of the administration of ulipristal acetate or a metabolite thereof.

The cycle of inhibitor and ulipristal acetate administration can be repeated one or more times, as appropriate to treat the patient.

Suitable ranges to be administered for the inhibitor, such as ketoconazole, include at least about 100 mg per day, at least about 200 mg per day, and at least about 300 mg per day. Examples of suitable ranges include from about 100 to 400 mg per day, about 200 to about 400 mg per day, and about 300 to about 400 mg per day.

The administration of the cytochrome p450 isozyme inhibitor may be once daily, or divided into two or more dosages given over a 24 hour period.

### Kits and Containers:

The invention provides various kits and containers.

In one embodiment, it is provided a kit comprising i) a dosage form comprising ulipristal acetate or a metabolite thereof, and (ii) printed matter stating that administration of ulipristal acetate or a metabolite thereof with a substance that affects activity of a cytochrome p450 isozyme can affect plasma concentration, safety, effectiveness or any combination thereof of ulipristal acetate or a metabolite thereof, the substance, or both.

A kit of particular interest comprises i) a dosage form comprising ulipristal acetate or a metabolite thereof, and (ii) printed matter stating that administration of an inducer of CYP3A4 enzyme may decrease plasma concentration of ulipristal acetate or a metabolite thereof, and/or that administration of an inducer of CYP3A4 enzyme may decrease effectiveness of ulipristal acetate or a metabolite thereof.

The printed matter usually serves as a labelling for the medicine. For instance it is conveniently a leaflet inserted into the packaging of the medicine, or it may be the packaging itself, on which the information is printed.

The examples illustrate the invention without limiting its scope.

### Example

### Ulipristal Acetate Inhibition of Cytochrome p450 Isozymes in Microsomes

Metabolism of ulipristal acetate was investigated in vitro in liver microsomal preparations from mice, rats, rabbits, dogs, monkeys and humans. All species produced the same two major metabolites but the proportions varied between species. No metabolite was detected which was unique to humans.

In studies with Supersome®, the metabolism of ulipristal acetate was predominantly mediated by CYP3A4.

Pharmacokinetic interaction studies in vitro examined the potential for ulipristal acetate to inhibit a range of CYP isozymes. Pooled human liver microsomes were incubated in the presence of 10 or 100 μM for the determination of metabolism of markers of specific cytochrome P450 isoenzyme activities—phenacetin O-deethylase (CYP1A2), tolbutamide methyl-hydroxylase (CYP2C9), S-mephenyloin 4-hydroxylase (CYP2C19), bufuralol 1-hydroxylase (CYP2D6), lauric acid 11-hydroxylase (CYP2E1) and midazolam 1-hydroxylase (CYP3A4). Positive control inhibitors for each isoenzyme were included.

Inhibition of CYP2C9, CYP2D6 and CYP3A4 was observed at high concentration (100 μM, 47.56 μg/mL).

1. A method of using ulipristal acetate or a metabolite thereof for providing contraception, comprising providing a patient with ulipristal acetate or a metabolite thereof, and informing the patient or a medical care worker that ulipristal acetate or a metabolite thereof affects activity of a cytochrome p450 isozyme, and that administration of ulipristal acetate or a metabolite thereof with a substance that affects activity of a cytochrome p450 isozyme can affect plasma concentration, safety, efficacy or any combination thereof of ulipristal acetate or a metabolite thereof, the substance, or both.

2. The method of claim 1, wherein the substance is a substrate of CYP3A4 isozyme.

3. The method of claim 1, wherein the substance is an inhibitor of CYP3A4 isozyme.

4. The method of claim 3, wherein said inhibitor is selected from the group consisting of ketoconazole, itraconazole, ritonavir, telitromycin, clarithromycin and nefazodone.

5. The method of claim 1, wherein the substrate is an inducer of CYP3A4.

6. A method for providing contraception in a female subject, comprising providing the subject with an amount of ulipristal acetate or a metabolite thereof, in combination with an inhibitor of CYP3A4 enzyme.

7. The method of claim 6, wherein the contraception is a post-coital contraception.

8. The method of claim 7 wherein post coital contraception is provided within about 120 hours after unprotected intercourse.

9. The method of claim 7, wherein the post coital contraception is an emergency contraception.

10. The method of claim 6, wherein the ulipristal acetate or a metabolite thereof is administered in an oral dosage form.

11. The method of claim 10 wherein the oral dosage form is a tablet.

12. The method of claim 6 wherein the dosage form comprises about 30 mg ulipristal acetate or a metabolite thereof.

13. The method of claim 6, wherein the ulipristal acetate or a metabolite thereof is administered in a form suitable for buccal, parenteral, transdermal, vaginal, or uterine route.

14. A method of using ulipristal acetate or a metabolite thereof for treating a patient's condition, comprising providing a patient with ulipristal acetate or a metabolite thereof, and informing the patient or a medical care worker that ulipristal acetate or a metabolite thereof affects activity of a cytochrome p450 isozyme, and that administration of ulipristal acetate or a metabolite thereof with a substance that affects activity of a cytochrome p450 isozyme can affect plasma concentration, safety, efficacy or any combination thereof of ulipristal acetate or a metabolite thereof, the substance, or both.

15. The method of claim 14, wherein the substance is a substrate of CYP3A4 isozyme.

16. The method of claim 14, wherein the substance is an inhibitor of CYP3A4 isozyme.

17. The method of claim 16, wherein said inhibitor is selected from the group consisting of ketoconazole, itraconazole, ritonavir, telitromycin, clarithromycin and nefazodone.

18. The method of claim 14, wherein the substrate is an inducer of CYP3A4.

19. The method of claim 14, wherein the condition is selected from the group consisting of endometriosis, dysmenorrhea, uterine leiomyoma (leiomyomata), uterine fibroid, excessive uterine bleeding (menorrhagia), either idiopathic or resulting from spontaneous or iatrogenic coagulation disorders, meningioma, hormonal diseases, such as hormone-responsive cancers, endocrine hormone-dependent tumors, breast cancer and inhibition of uterine endometrial proliferation.

20. The method of claim 19, wherein the condition is uterine leiomyoma.

21. A method for treating a patient's condition, comprising providing the patient with an effective amount of ulipristal acetate or a metabolite thereof, in combination with an inhibitor of CYP3A4 enzyme.

22. The method of claim 21, wherein the condition is selected from the group consisting of endometriosis, dysmenorrhea, uterine leiomyoma (leiomyomata), uterine fibroid, excessive uterine bleeding (menorrhagia), either idiopathic or resulting from spontaneous or iatrogenic coagulation disorders, meningioma, hormonal diseases, such as hormone-responsive cancers, endocrine hormone-dependent tumors, breast cancer and inhibition of uterine endometrial proliferation.

23. The method of claim 22, wherein the condition is uterine leiomyoma.

24. The method of claim 21, wherein said inhibitor is selected from the group consisting of ketoconazole, itraconazole, ritonavir, telitromycin, clarithromycin and nefazodone.

25. A pharmaceutical composition comprising ulipristal acetate or a metabolite thereof and an inhibitor of CYP3A4 enzyme, in association with a pharmaceutically acceptable carrier.

26. The pharmaceutical composition of claim 25, wherein said inhibitor is selected from the group consisting of ketoconazole, itraconazole, ritonavir, telitromycin, clarithromycin and nefazodone.

27. A kit comprising i) a dosage form comprising ulipristal acetate or a metabolite thereof and ii) a dosage form comprising an inhibitor of CYP3A4 enzyme.

28. The kit of claim 27, wherein said inhibitor is selected from the group consisting of ketoconazole, itraconazole, ritonavir, telitromycin, clarithromycin and nefazodone.

29. The kit of claim 27, wherein the dosage form is an oral dosage form.

30. The kit of claim 29, wherein the oral dosage form is a tablet.

31. The kit of claim 27, wherein the dosage form comprises about 30 mg ulipristal acetate or a metabolite thereof.

32. A kit comprising i) a dosage form comprising ulipristal acetate or a metabolite thereof, and (ii) printed matter stating that administration of ulipristal acetate or a metabolite thereof with a substance that affects activity of a cytochrome p450 isozyme can affect plasma concentration, safety, effectiveness or any combination thereof of ulipristal acetate or a metabolite thereof, the substance, or both

33. A kit comprising i) a dosage form comprising ulipristal acetate or a metabolite thereof, and (ii) printed matter stating that administration of an inducer of CYP3A4 enzyme may decrease plasma concentration of ulipristal acetate or a metabolite thereof, and/or that administration of an inducer of CYP3A4 enzyme may decrease effectiveness of ulipristal acetate or a metabolite thereof.
